(19) United States
(12) Patent Application Publication
(10) Pub. No.: US 2022/0399403 A1
(43) Pub. Date: Dec. 15, 2022

(54) DETECTION DEVICE

(71) Applicant: Japan Display Inc., Tokyo (JP)

(72) Inventors: Isao SUZUMURA, Tokyo (JP); Kento HIMOTO, Tokyo (JP); Takashi NAKAMURA, Tokyo (JP); Masahiro TADA, Tokyo (JP); Yasushi TOMIOKA, Tokyo (JP)

(73) Assignee: Japan Display Inc., Tokyo (JP)

(21) Appl. No.: 17/838,314

(22) Filed: Jun. 13, 2022

(30) Foreign Application Priority Data
Jun. 14, 2021 (JP) 2021-098908

(51) Int. Cl. H01L 27/30; H01L 51/42; H01L 51/44

(52) U.S. Cl. CPC H01L 27/307; H01L 51/4253; H01L 51/4293; H01L 51/441; A61B 5/1172

(57) ABSTRACT

A photo detecting device, includes a plurality of photodiodes arranged above a substrate, a lower electrode and a first inorganic insulating film that are provided between the substrate and the photodiodes in a direction orthogonal to a surface of the substrate, and an upper electrode provided above the photodiodes. Each of the photodiodes comprises an active layer, a first carrier transport layer provided between the active layer and the lower electrode, and a second carrier transport layer provided between the active layer and the upper electrode, the first inorganic insulating film is provided between the lower electrode and the first carrier transport layer, and the first inorganic insulating film covers at least an end on an outer edge side of the lower electrode.

### CROSS-REFERENCE TO RELATED APPLICATION

This application claims the benefit of priority from Japanese Patent Application No. 2021-098908 filed on Jun. 14, 2021, the entire contents of which are incorporated herein by reference.

### BACKGROUND

### 1. Technical Field

The present disclosure relates to a detection device.

### 2. Description of the Related Art

Optical sensors capable of detecting fingerprint patterns and vein patterns are known (for example, Japanese Patent Application Laid-open Publication No. 2009-032005). Such optical sensors each include a plurality of photodiodes each including an organic semiconductor material used as an active layer. Each of the photodiodes is disposed between a lower electrode and an upper electrode, and the lower electrode, an electron transport layer, the active layer, a hole transport layer, and the upper electrode are stacked in this order.

In areas where the electron transport layer (or the hole transport layer) disposed between the active layer and the lower electrode is thinly formed and the coverage of the electron transport layer is insufficient, a short circuit may occur between the active layer and the lower electrode.

It is an object of the present disclosure to provide a detection device capable of reducing the occurrence of the short circuit between the active layer and the lower electrode.

### SUMMARY

A photo detecting device according to an embodiment of the present disclosure includes a plurality of photodiodes arranged above a substrate, a lower electrode and a first inorganic insulating film that are provided between the substrate and the photodiodes in a direction orthogonal to a surface of the substrate, and an upper electrode provided above the photodiodes. Each of the photodiodes comprises an active layer, a first carrier transport layer provided between the active layer and the lower electrode, and a second carrier transport layer provided between the active layer and the upper electrode, the first inorganic insulating film is provided between the lower electrode and the first carrier transport layer, and the first inorganic insulating film covers at least an end on an outer edge side of the lower electrode.

### BRIEF DESCRIPTION OF THE DRAWINGS

FIG. 1 is a plan view illustrating a detection device according to an embodiment of the present disclosure;

FIG. 2 is a block diagram illustrating a configuration example of the detection device according to the embodiment;

FIG. 3 is a circuit diagram illustrating the detection device;

FIG. 4 is a circuit diagram illustrating a plurality of partial detection areas;

FIG. 5 is a magnified schematic configuration diagram of a sensor unit;

FIG. 6 is a VI-VI′ sectional view of FIG. 5;

FIG. 7 is a magnified schematic sectional view illustrating a magnified view of a layered structure of a first inorganic insulating film, a lower electrode, a photodiode, and an upper electrode near a first contact hole in FIG. 6;

FIG. 8 is a sectional view illustrating a detection device according to a first modification; and

FIG. 9 is a sectional view illustrating a detection device according to a second modification.

### DETAILED DESCRIPTION

The following describes a mode (embodiment) for carrying out the present disclosure in detail with reference to the drawings. The present disclosure is not limited by the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present disclosure and the drawings, and detailed description thereof may not be repeated where appropriate.

In the present specification and claims, in expressing an aspect of disposing another structure above a certain structure, a case of simply expressing “above” includes both a case of disposing the other structure immediately above the certain structure so as to contact the certain structure and a case of disposing the other structure above the certain structure with still another structure interposed therebetween, unless otherwise specified.

Embodiment

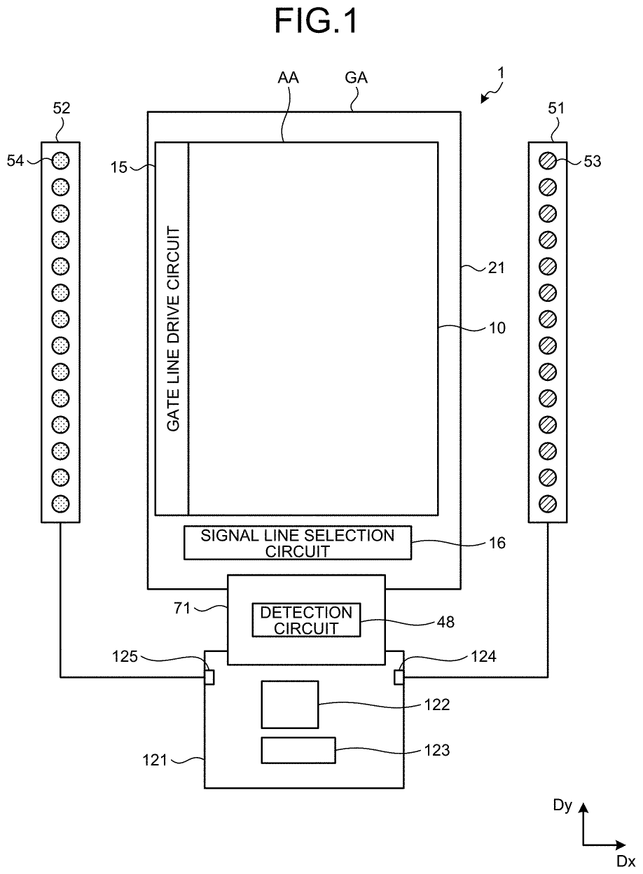

FIG. 1 is a plan view illustrating a detection device according to an embodiment of the present disclosure. As illustrated in FIG. 1, a detection device 1 includes a sensor base material 21, a sensor unit 10, a gate line drive circuit 15, a signal line selection circuit 16, a detection circuit 48, a control circuit 122, a power supply circuit 123, a first light source base material 51, a second light source base material 52, first light sources 53, and second light sources 54. The first light source base material 51 is provided with the first light sources 53. The second light source base material 52 is provided with the second light sources 54.

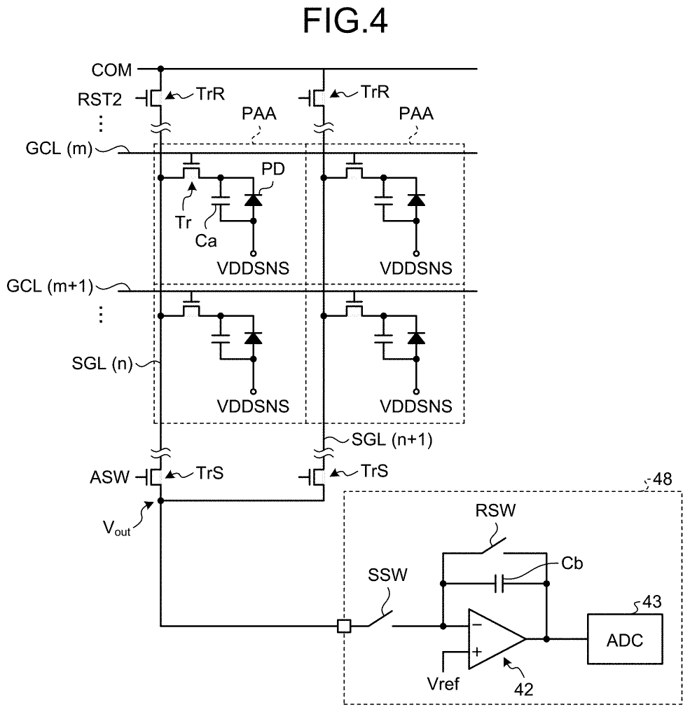

The sensor base material 21 is electrically coupled to a control substrate 121 through a flexible printed circuit board 71. The flexible printed circuit board 71 is provided with the detection circuit 48. The control substrate 121 is provided with the control circuit 122 and the power supply circuit 123. The control circuit 122 is, for example, a field-programmable gate array (FPGA). The control circuit 122 supplies control signals to the sensor unit 10, the gate line drive circuit 15, and the signal line selection circuit 16 to control a detection operation of the sensor unit 10. The control circuit 122 supplies control signals to the first and the second light sources 53 and 54 to control lighting or non-lighting of the first and the second light sources 53 and 54. The power supply circuit 123 supplies voltage signals including, for example, a sensor power supply signal VDDSNS (refer to FIG. 4) to the sensor unit 10, the gate line drive circuit 15, and the signal line selection circuit 16. The power supply circuit 123 supplies a power supply voltage to the first and the second light sources 53 and 54.

The sensor base material 21 has a detection area AA and a peripheral area GA. The detection area AA is an area provided with a plurality of photodiodes PD (refer to FIG. 4) included in the sensor unit 10. The peripheral area GA is an area between the outer perimeter of the detection area AA and ends of the sensor base material 21, and is an area not provided with the photodiodes PD.

The gate line drive circuit 15 and the signal line selection circuit 16 are provided in the peripheral area GA. Specifically, the gate line drive circuit 15 is provided in an area extending along a second direction Dy in the peripheral area GA. The signal line selection circuit 16 is provided in an area extending along a first direction Dx in the peripheral area GA, and is provided between the sensor unit 10 and the detection circuit 48.

The first direction Dx is one direction in a plane parallel to the sensor base material 21. The second direction Dy is one direction in the plane parallel to the sensor base material 21, and is a direction orthogonal to the first direction Dx. The second direction Dy may non-orthogonally intersect the first direction Dx. The term “plan view” refers to a positional relation when viewed from a direction orthogonal to the sensor base material 21.

The first light sources 53 are provided on the first light source base material 51, and are arranged along the second direction Dy. The second light sources 54 are provided on the second light source base material 52, and are arranged along the second direction Dy. The first and the second light source base materials 51 and 52 are electrically coupled to the control circuit 122 and the power supply circuit 123, respectively, through terminals 124 and 125 provided on the control substrate 121.

For example, inorganic light-emitting diodes (LEDs) or organic electroluminescence (EL) devices (organic light-emitting diodes: OLEDs) are used as the first and the second light sources 53 and 54. The first and the second light sources 53 and 54 emit first and second light, respectively, having different wavelengths.

The first light emitted from the first light sources 53 is mainly reflected on a surface of an object to be detected such as a finger, and is incident on the sensor unit 10. As a result, the sensor unit 10 can detect a fingerprint by detecting a shape of asperities on the surface of the finger or the like. The second light emitted from the second light sources 54 is mainly reflected in the finger or the like, or transmitted through the finger or the like, and is incident on the sensor unit 10. As a result, the sensor unit 10 can detect information on a living body in the finger or the like. Examples of the information on the living body include a pulse wave, pulsation, and a vascular image of the finger or a palm. That is, the detection device 1 may be configured as a fingerprint detection device to detect the fingerprint or a vein detection device to detect a vascular pattern of, for example, veins.

The first light may have a wavelength of from 500 nm to 600 nm, for example, a wavelength of approximately 550 nm, and the second light may have a wavelength of from 780 nm and 950 nm, for example, a wavelength of approximately 850 nm. In this case, the first light is blue or green visible light, and the second light is infrared light. The sensor unit 10 can detect the fingerprint based on the first light emitted from the first light sources 53. The second light emitted from the second light sources 54 is reflected in the object to be detected such as the finger, or transmitted through or absorbed by the finger or the like, and is incident on the sensor unit 10. As a result, the sensor unit 10 can detect the pulse wave or the vascular image (vascular pattern) as the information on the living body in the finger or the like.

Alternatively, the first light may have a wavelength of from 600 nm to 700 nm, for example, approximately 660 nm, and the second light may have a wavelength of from 780 nm and 900 nm, for example, approximately 850 nm. In this case, the sensor unit 10 can detect a blood oxygen saturation level in addition to the pulse wave, the pulsation, and the vascular image as the information on the living body based on the first light emitted from the first light sources 53 and the second light emitted from the second light sources 54. Thus, the detection device 1 includes the first and the second light sources 53 and 54, and therefore, can detect the various information on the living body by performing the detection based on the first light and the detection based on the second light.

The arrangement of the first and the second light sources 53 and 54 illustrated in FIG. 1 is merely an example, and may be changed as appropriate. The detection device 1 is provided with a plurality of types of light sources (first and second light sources 53 and 54) as the light sources. However, the light sources are not limited thereto, and may be of one type. For example, the first and the second light sources 53 and 54 may be arranged on each of the first and the second light source base materials 51 and 52. The first and the second light sources 53 and 54 may be provided on one or three or more light source base materials. In other words, at least one light source needs to be disposed.

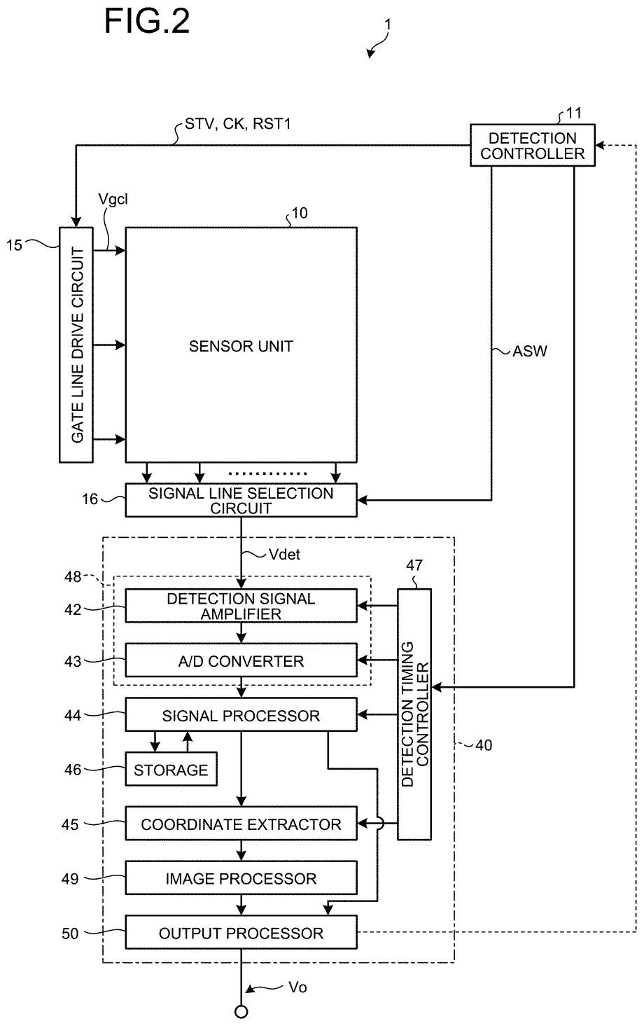

FIG. 2 is a block diagram illustrating a configuration example of the detection device according to the embodiment. As illustrated in FIG. 2, the detection device 1 further includes a detection controller 11 and a detector 40. The control circuit 122 includes one, some, or all functions of the detection controller 11. The control circuit 122 also includes one, some, or all functions of the detector 40 except those of the detection circuit 48.

The sensor unit 10 includes the photodiodes PD. Each of the photodiodes PD included in the sensor unit 10 outputs an electrical signal corresponding to light irradiating the photodiode PD as a detection signal Vdet to the signal line selection circuit 16. The sensor unit 10 performs the detection according to a gate drive signal Vgcl supplied from the gate line drive circuit 15.

The detection controller 11 is a circuit that supplies respective control signals to the gate line drive circuit 15, the signal line selection circuit 16, and the detector 40 to control operations thereof. The detection controller 11 supplies various control signals such as a start signal STV, a clock signal CK, and a reset signal RST1 to the gate line drive circuit 15. The detection controller 11 also supplies various control signals such as a selection signal ASW to the signal line selection circuit 16. The detection controller 11 supplies various control signals to the first and the second light sources 53 and 54 to control the lighting and non-lighting of the respective first and second light sources 53 and 54.

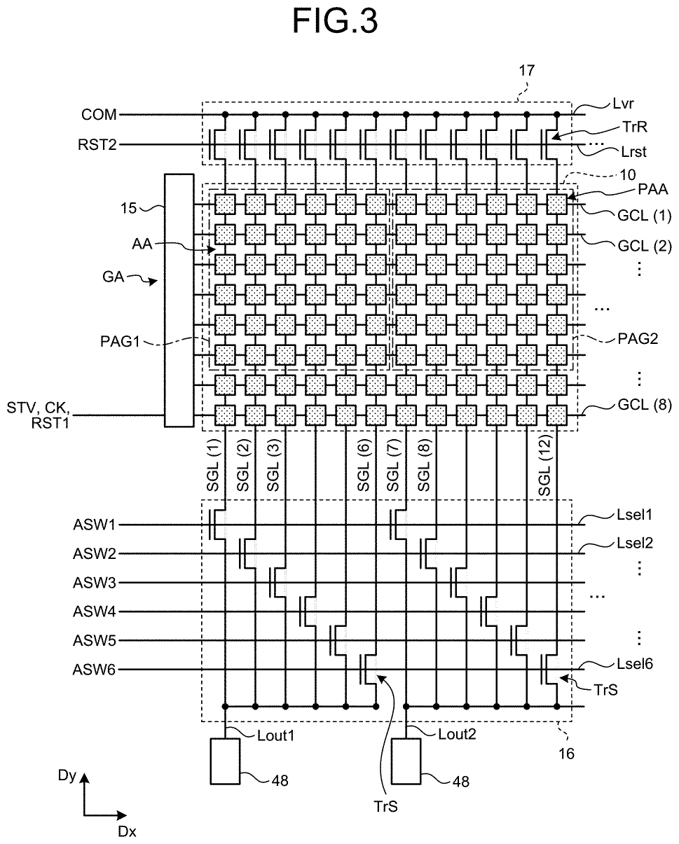

The gate line drive circuit 15 is a circuit that drives a plurality of gate lines GCL (refer to FIG. 3) based on the various control signals. The gate line drive circuit 15 sequentially or simultaneously selects the gate lines GCL, and supplies the gate drive signals Vgcl to the selected gate lines GCL. Through this operation, the gate line drive circuit 15 selects the photodiodes PD coupled to the gate lines GCL.

The signal line selection circuit 16 is a switch circuit that sequentially or simultaneously selects a plurality of signal lines SGL (refer to FIG. 3). The signal line selection circuit 16 is, for example, a multiplexer. The signal line selection circuit 16 couples the selected signal lines SGL to the detection circuit 48 based on the selection signal ASW supplied from the detection controller 11. Through this operation, the signal line selection circuit 16 outputs the detection signals Vdet of the photodiodes PD to the detector 40.

The detector 40 includes the detection circuit 48, a signal processor 44, a coordinate extractor 45, a storage 46, a detection timing controller 47, an image processor 49, and an output processor 50. Based on a control signal supplied from the detection controller 11, the detection timing controller 47 controls the detection circuit 48, the signal processor 44, the coordinate extractor 45, and the image processor 49 so as to operate in synchronization with one another.

The detection circuit 48 is, for example, an analog front-end (AFE) circuit. The detection circuit 48 is a signal processing circuit having functions of at least a detection signal amplifier 42 and an analog-to-digital (A/D) converter 43. The detection signal amplifier 42 amplifies the detection signals Vdet. The A/D converter 43 converts analog signals output from the detection signal amplifier 42 into digital signals.

The signal processor 44 is a logic circuit that detects a predetermined physical quantity received by the sensor unit 10 based on an output signal of the detection circuit 48. When the finger is in contact with or in proximity to a detection surface, the signal processor 44 can detect the asperities on the surface of the finger or the palm based on the signal from the detection circuit 48. The signal processor 44 can also detect the information on the living body based on the signal from the detection circuit 48. Examples of the information on the living body include the vascular image, the pulse wave, the pulsation, and the blood oxygen level of the finger or the palm.

The signal processor 44 may also perform processing of acquiring the detection signals Vdet (information on the living body) simultaneously detected by the photodiodes PD, and averaging the detection signals Vdet. In this case, the detector 40 can perform stable detection by reducing measurement errors caused by noise or relative positional misalignment between the object to be detected, such as the finger, and the sensor unit 10.

The storage 46 temporarily stores therein a signal calculated by the signal processor 44. The storage 46 may be, for example, a random-access memory (RAM) or a register circuit.

The coordinate extractor 45 is a logic circuit that obtains detected coordinates of the asperities on the surface of the finger or the like when the contact or the proximity of the finger is detected by the signal processor 44. The coordinate extractor 45 is also a logic circuit that obtains detected coordinates of blood vessels of the finger or the palm. The image processor 49 combines the detection signals Vdet output from the respective photodiodes PD of the sensor unit 10 to generate two-dimensional information representing the shape of the asperities on the surface of the finger or the like and two-dimensional information representing the shape of the blood vessels of the finger or the palm. The coordinate extractor 45 may output the detection signals Vdet as sensor output voltages Vo instead of calculating the detected coordinates. A case can be considered where the detector 40 does not include the coordinate extractor 45 and the image processor 49.

The output processor 50 serves as a processor that performs processing based on the outputs from the photodiodes PD. The output processor 50 may include, for example, the detected coordinates obtained by the coordinate extractor 45 and the two-dimensional information generated by the image processor 49 in the sensor output voltages Vo. The function of the output processor 50 may be integrated into another component (such as the image processor 49).

The following describes a circuit configuration example of the detection device 1. FIG. 3 is a circuit diagram illustrating the detection device. As illustrated in FIG. 3, the sensor unit 10 has a plurality of partial detection areas PAA arranged in a matrix having a row-column configuration. Each of the partial detection areas PAA is provided with the photodiode PD.

The gate lines GCL extend in the first direction Dx, and are coupled to the partial detection areas PAA arranged in the first direction Dx. A plurality of gate lines GCL(1), GCL(2), . . . , GCL(8) are arranged in the second direction Dy, and are each coupled to the gate line drive circuit 15. In the following description, the gate lines GCL(1), GCL(2), . . . , GCL(8) will each be simply referred to as the gate line GCL when need not be distinguished from one another. For ease of understanding of the description, FIG. 3 illustrates eight of the gate lines GCL. However, this is merely an example, and M (where M is eight or larger, and is, for example, 256) of the gate lines GCL may be arranged.

The signal lines SGL extend in the second direction Dy, and are coupled to the photodiodes PD of the partial detection areas PAA arranged in the second direction Dy. A plurality of signal lines SGL(1), SGL(2), . . . , SGL(12) are arranged in the first direction Dx, and are each coupled to the signal line selection circuit 16 and a reset circuit 17. In the following description, the signal lines SGL(1), SGL(2), . . . , SGL(12) will each be simply referred to as the signal line SGL when need not be distinguished from one another.

For ease of understanding of the description, 12 of the signal lines SGL are illustrated. However, this is merely an example, and N (where N is 12 or larger, and is, for example, 252) of the signal lines SGL may be arranged. The resolution of the sensor is, for example, 508 dots per inch (dpi), and the number of cells is 252×256. In FIG. 3, the sensor unit 10 is provided between the signal line selection circuit 16 and the reset circuit 17. The present disclosure is not limited thereto. The signal line selection circuit 16 and the reset circuit 17 may be coupled to ends in the same direction of the signal lines SGL.

The gate line drive circuit 15 receives the various control signals such as the start signal STV, the clock signal CK, and the reset signal RST1 from the control circuit 122 (refer to FIG. 1). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and corresponding ones of the partial detection areas PAA arranged in the first direction Dx are selected as detection targets.

The signal line selection circuit 16 includes a plurality of selection signal lines Lsel, a plurality of output signal lines Lout, and third switching elements TrS. The third switching elements TrS are provided correspondingly to the respective signal lines SGL. Six of the signal lines SGL(1), SGL(2), SGL(6) are coupled to a common output signal line Lout1. Six of the signal lines SGL(7), SGL(8), SGL(12) are coupled to a common output signal line Lout2. The output signal lines Lout1 and Lout2 are each coupled to the detection circuit 48.

The signal lines SGL(1), SGL(2), SGL(6) are grouped into a first signal line block, and the signal lines SGL(7), SGL(8), SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

The control circuit 122 (refer to FIG. 1) sequentially supplies the selection signal ASW to the selection signal lines Lsel. As a result, through the operations of the third switching elements TrS, the signal line selection circuit 16 sequentially selects the signal lines SGL in one of the signal line blocks in a time-division manner. The signal line selection circuit 16 selects one of the signal lines SGL in each of the signal line blocks. With the above-described configuration, the detection device 1 can reduce the number of integrated circuits (ICs) including the detection circuit 48 or the number of terminals of the ICs. The signal line selection circuit 16 may couple a plurality of the signal lines SGL in a bundle to the detection circuit 48.

As illustrated in FIG. 3, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 4) included in each of the partial detection areas PAA.

FIG. 4 is a circuit diagram illustrating the partial detection areas. FIG. 4 also illustrates a circuit configuration of the detection circuit 48. As illustrated in FIG. 4, each of the partial detection areas PAA includes the photodiode PD, the capacitive element Ca, and a corresponding one of the first switching elements Tr. The capacitive element Ca is capacitance (sensor capacitance) generated in the photodiode PD, and is equivalently coupled in parallel with the photodiode PD.

FIG. 4 illustrates two gate lines GCL(m) and GCL(m+1) arranged in the second direction Dy among the gate lines GCL. FIG. 4 also illustrates two signal lines SGL(n) and SGL(n+1) arranged in the first direction Dx among the signal lines SGL. The partial detection area PAA is an area surrounded by the gate lines GCL and the signal lines SGL.

Each of the first switching elements Tr is provided correspondingly to the photodiode PD. The first switching element Tr is constituted by a thin-film transistor, and in this example, constituted by an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the photodiode PD and the capacitive element Ca.

The anode of the photodiode PD is supplied with the sensor power supply signal VDDSNS from the power supply circuit 123. The signal line SGL and the capacitive element Ca are supplied with the reference signal COM that serves as an initial potential of the signal line SGL and the capacitive element Ca from the power supply circuit 123.

When the partial detection area PAA is irradiated with light, a current corresponding to the amount of the light flows through the photodiode PD. As a result, an electrical charge is stored in the capacitive element Ca. After the first switching element Tr is turned on, a current corresponding to the electrical charge stored in the capacitive element Ca flows through the signal line SGL. The signal line SGL is coupled to the detection circuit 48 through a corresponding one of the third switching elements TrS of the signal line selection circuit 16. Thus, the detection device 1 can detect a signal corresponding to the amount of the light irradiating the photodiode PD in each of the partial detection areas PAA or each block unit PAG.

During a reading period, a switch SSW of the detection circuit 48 is turned on, and the detection circuit 48 is coupled to the signal lines SGL. The detection signal amplifier 42 of the detection circuit 48 converts a variation of a current supplied from the signal lines SGL into a variation of a voltage, and amplifies the result. A reference potential (Vref) having a fixed potential is supplied to a non-inverting input portion (+) of the detection signal amplifier 42, and the signal lines SGL are coupled to an inverting input terminal (−) of the detection signal amplifier 42. In the embodiment, the same signal as the reference signal COM is supplied as the reference potential (Vref). The signal processor 44 (refer to FIG. 2) calculates the difference between the detection signal Vdet when light irradiates the photodiode PD and the detection signal Vdet when light does not irradiate the photodiode PD as each of the sensor output voltages Vo. The detection signal amplifier 42 includes a capacitive element Cb and a reset switch RSW. During a reset period, the reset switch RSW is turned on, and an electrical charge of the capacitive element Cb is reset.

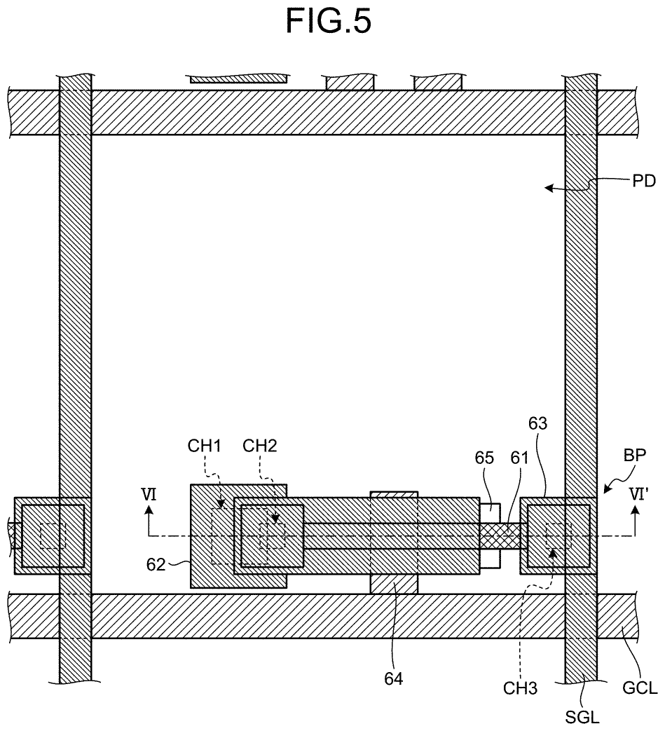

The following describes a configuration of the photodiode PD. FIG. 5 is a magnified schematic configuration diagram of the sensor unit. As illustrated in FIG. 5, the photodiode PD is formed on a backplane BP. The backplane BP is a drive circuit board for driving the photodiode PD, and is also called an array substrate or an active matrix substrate.

The backplane BP includes the sensor base material 21, various transistors, such as the first switching elements Tr, formed on the sensor base material 21, and various types of wiring such as the gate lines GCL and the signal lines SGL.

The first switching element Tr includes a semiconductor layer 61, a source electrode 62, a drain electrode 63, and a gate electrode 64. The semiconductor layer 61 extends along the gate line GCL, and is provided so as to intersect the gate electrode 64 in the plan view. The gate electrode 64 is coupled to the gate line GCL, and extends in a direction orthogonal to the gate line GCL. One end side of the semiconductor layer 61 is coupled to the source electrode 62 through a second contact hole CH2. The source electrode 62 is electrically coupled to the photodiode PD through a first contact hole CH1. The other end side of the semiconductor layer 61 is coupled to the drain electrode 63 through a third contact hole CH3. The drain electrode 63 is coupled to the signal line SGL.

The configuration and the arrangement of the first switching element Tr illustrated in FIG. 5 are merely exemplary, and can be changed as appropriate.

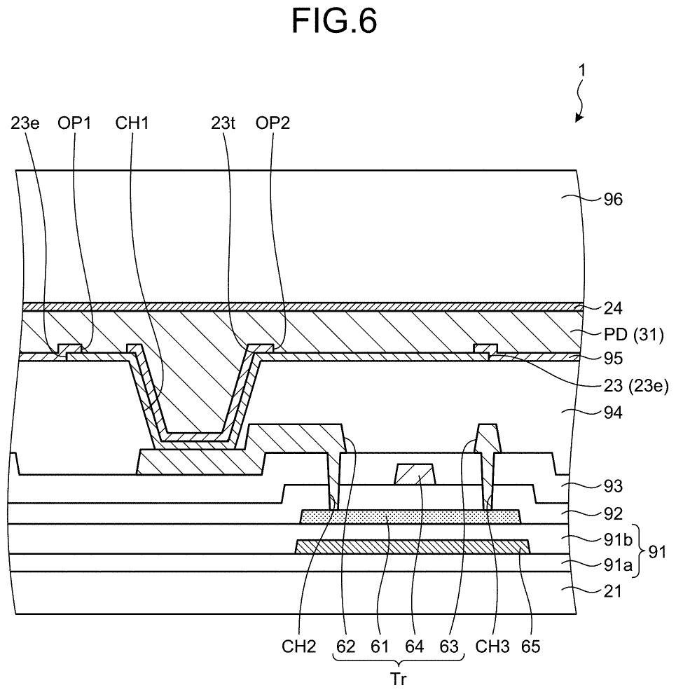

FIG. 6 is a VI-VI′ sectional view of FIG. 5. As illustrated in FIG. 6, the detection device 1 includes the sensor base material 21, the first switching element Tr, an organic insulating film 94, a lower electrode 23, a first inorganic insulating film 95, the photodiode PD (FIG. 6 illustrates only an active layer 31), an upper electrode 24, and a sealing film 96.

The sensor base material 21 is an insulating base material, and is made using, for example, glass or a resin material. The sensor base material 21 is not limited to having a flat plate shape, and may have a curved surface. In this case, the sensor base material 21 can be a film-like resin.

In this specification, a direction from the sensor base material 21 toward the photodiode PD in a direction orthogonal to a surface of the sensor base material 21 is referred to as “upper side” or simply “above”. A direction from the photodiode PD toward the sensor base material 21 is referred to as “lower side” or simply “below”.

An undercoat film 91 is provided above the sensor base material 21. The undercoat film 91 is, for example, a two-layered structure having insulating films 91a and 91b. The undercoat film 91 is formed of, for example, an inorganic insulating film such as a silicon nitride film or a silicon oxide film. The configuration of the undercoat film 91 is not limited to that illustrated in FIG. 6. For example, the undercoat film 91 may be a single layer film, or may have three or more layers.

A light-blocking film 65 is provided above the insulating film 91a. The light-blocking film 65 is provided between the semiconductor layer 61 and the sensor base material 21. The detection device 1 (photodiode PD) of the present embodiment is a bottom-surface light receiving optical sensor, and the light reflected on the surface of the object to be detected such as the finger is incident on the photodiode PD from the bottom surface side of the sensor base material 21. The light-blocking film 65 can restrain light from entering a channel area of the semiconductor layer 61 from the sensor base material 21 side.

The first switching element Tr (transistor) is provided above the sensor base material 21. The semiconductor layer 61 is provided above the undercoat film 91. For example, polysilicon is used as the semiconductor layer 61. The semiconductor layer 61 is, however, not limited thereto, and may be formed of, for example, a microcrystalline oxide semiconductor, an amorphous oxide semiconductor, or low-temperature polysilicon. Although only the n-type TFT is illustrated as the first switching element Tr, a p-type TFT may be formed at the same time.

A gate insulating film 92 is provided above the undercoat film 91 so as to cover the semiconductor layer 61. The gate insulating film 92 is, for example, an inorganic insulating film such as a silicon oxide film. The gate electrode 64 is provided above the gate insulating film 92. In the example illustrated in FIG. 6, the first switching element Tr has a top-gate structure. However, the first switching element Tr is not limited thereto, and may have a bottom-gate structure, or a dual-gate structure in which the gate electrodes 64 are provided both above and below the semiconductor layer 61.

An interlayer insulating film 93 is provided above the gate insulating film 92 so as to cover the gate electrode 64. The interlayer insulating film 93 has, for example, a multilayered structure of a silicon nitride film and a silicon oxide film. The source electrode 62 and the drain electrode 63 are provided above the interlayer insulating film 93. The source electrode 62 is coupled to a source area of the semiconductor layer 61 through the second contact hole CH2 provided in the gate insulating film 92 and the interlayer insulating film 93. The drain electrode 63 is coupled to a drain area of the semiconductor layer 61 through the third contact hole CH3 provided in the gate insulating film 92 and the interlayer insulating film 93.

The organic insulating film 94 is provided above the interlayer insulating film 93 so as to cover the source electrode 62 and the drain electrode 63 of the first switching element Tr. The organic insulating film 94 is an organic planarizing film, and has a better coverage property for wiring steps and provides better surface flatness than inorganic insulating materials formed by, for example, chemical vapor deposition (CVD).

The photodiode PD is provided above the organic insulating film 94. The lower electrode 23 and the first inorganic insulating film 95 are provided between both the sensor base material 21 and the organic insulating film 94 and the photodiode PD in a direction orthogonal to the surface of the sensor base material 21.

In more detail, the lower electrode 23 is provided above the organic insulating film 94, and is coupled to the source electrode 62 of the first switching element Tr on the bottom surface of the first contact hole CH1 formed in the organic insulating film 94. The lower electrode 23 is a cathode electrode of the photodiode PD, and is formed of a light-transmitting conductive material such as indium tin oxide (ITO) or indium zinc oxide (IZO). The lower electrodes 23 are arranged so as to be separated for the respective partial detection areas PAA (photodiodes PD). The photodiode PD has a larger area than that of the lower electrode 23 in the plan view, and covers the upper surface and an end 23e on an outer edge side of the lower electrode 23.

The first inorganic insulating film 95 is provided above the organic insulating film 94 so as to cover the lower electrode 23. A material such as a silicon nitride film or an aluminum oxide film is used as the first inorganic insulating film 95. The first inorganic insulating film 95 covers the upper surface of the lower electrode 23, and has at least one or more openings (first opening OP1 and second opening OP2) in an area overlapping the upper surface of the lower electrode 23. The photodiode PD is electrically coupled to the lower electrode 23 through the first and the second openings OP1 and OP2. The number of the first and the second openings OP1 and OP2 provided in the first inorganic insulating film 95 is not limited to two. The first inorganic insulating film 95 only needs to be provided with at least one opening, and may be provided with three or more openings.

The first inorganic insulating film 95 has a larger area than that of the lower electrode 23 in the plan view, and covers at least the end 23e on the outer edge side of the lower electrode 23. The first inorganic insulating film 95 is provided in an area overlapping the photodiode PD, and is provided between the organic insulating film 94 and the photodiode PD in an area not overlapping the lower electrode 23. With this configuration, the first inorganic insulating film 95 also serves as a barrier film that restrains water from entering the photodiode PD from the organic insulating film 94.

The first inorganic insulating film 95 is also formed in the first contact hole CH1. The lower electrode 23 and the first inorganic insulating film 95 are stacked on the inner side surface and the bottom surface of the first contact hole CH1. The organic insulating film 94, the lower electrode 23, and the first inorganic insulating film 95 are stacked in this order on the inner side surface of the first contact hole CH1. The source electrode 62, the lower electrode 23, and the first inorganic insulating film 95 are stacked in this order on the bottom surface of the first contact hole CH1. The first inorganic insulating film 95 is provided so as to cover a corner 23t of the lower electrode 23 in a position overlapping the open end of the first contact hole CH1.

The upper electrode 24 is provided above the photodiode PD. The upper electrode 24 is an anode electrode of the photodiode PD, and is continuously formed over the partial detection areas PAA (photodiodes PD). The upper electrode 24 is made using a metal material such as silver (Ag), and serves as a reflective electrode.

The sealing film 96 is provided above the upper electrode 24. An inorganic film, such as a silicon nitride film or an aluminum oxide film, or a resin film, such as an acrylic film, is used as the sealing film 96. The sealing film 96 is not limited to a single layer, and may be a multilayered film of two or more layers obtained by combining the inorganic film with the resin film described above. The sealing film 96 well seals the photodiode PD, and thus can restrain water from entering the photodiode PD from the upper surface side.

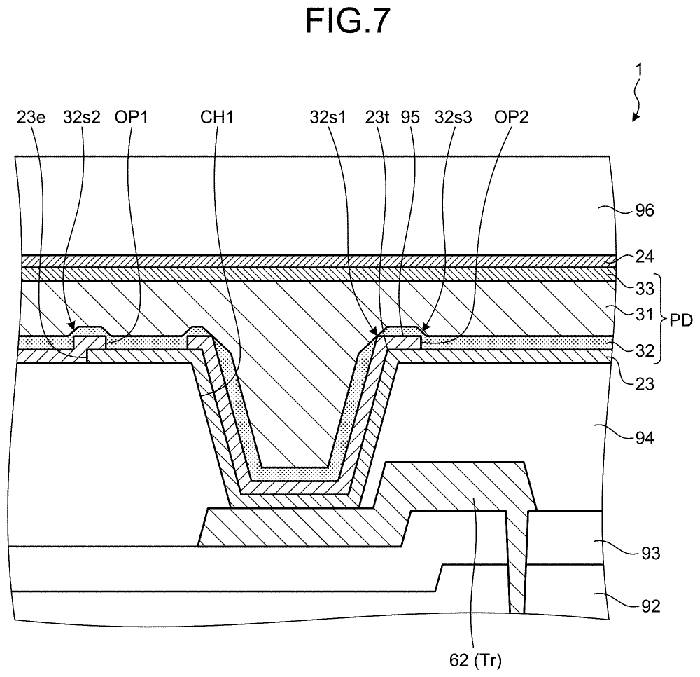

The following describes a detailed layered structure of the first inorganic insulating film 95, the lower electrode 23, the photodiode PD, and the upper electrode 24. FIG. 7 is a magnified schematic sectional view illustrating a magnified view of the layered structure of the first inorganic insulating film, the lower electrode, the photodiode, and the upper electrode near the first contact hole in FIG. 6. As illustrated in FIG. 7, the photodiode PD includes the active layer 31, an electron transport layer 32 (first carrier transport layer) provided between the active layer 31 and the lower electrode 23, and a hole transport layer 33 (second carrier transport layer) provided between the active layer 31 and the upper electrode 24.

In the detection device 1, the organic insulating film 94, the lower electrode 23, the first inorganic insulating film 95, the electron transport layer 32, the active layer 31, the hole transport layer 33, and the upper electrode 24 are stacked in this order in the direction orthogonal to the sensor base material 21.

The electron transport layer 32 is formed by coating using a material such as zinc acetate, ethoxylated polyethylenimine (PEIE), or polyethylenimine (PEI). The electron transport layer 32 is a single layer, and has a thickness of, for example, approximately 30 nm or smaller.

A mixture of a p-type organic semiconductor and an n-type organic semiconductor is used as the active layer 31. PMDPP3T(poly((2,5-bis(2-hexyldecyl)-2,3,5,6-tetrahydro-3,6-dioxopyrrolo(3,4-c)pyrrole-1,4-diyl)-alt-(3′,3″-dimethyl-2,2′:5′,2″-terthiophene)-5,5″-diyl)) is an example of the p-type organic semiconductor. PC61BM([6,6]-phenyl C61-butyric acid methyl ester) is an example of the n-type organic semiconductor. The thickness of the active layer 31 is, for example, approximately from 100 nm to 500 nm, and preferably approximately 350 nm.

The hole transport layer 33 is, for example, a metal oxide layer of, for example, tungsten oxide (WO₃) or a molybdenum oxide (MoOx). The hole transport layer 33 is formed of a vapor-deposited film or a sputtered film, and has a thickness of, for example, approximately 30 nm or smaller.

The thickness of the lower electrode 23 is, for example, approximately 50 nm. The thickness of the upper electrode 24 is, for example, approximately 100 nm or smaller. That is, the thickness of each of the electron transport layer 32 and the hole transport layer 33 is smaller than that of the active layer 31 and smaller than that of the lower electrode 23 and the upper electrode 24. In other words, the thickness of each of the lower electrode 23 and the upper electrode 24 is smaller than that of the active layer 31 and larger than that of the electron transport layer 32 and the hole transport layer 33.

The materials and the manufacturing methods of the electron transport layer 32, the active layer 31, and the hole transport layer 33 are only examples, and other materials and manufacturing methods may be used. For example, the electron transport layer 32 may be a vapor-deposited film or a sputtered film using a material such as zinc oxide (ZnO) or a titanium oxide (TiO₂) . The hole transport layer 33 may be a vapor-deposited film or a sputtered film using a material such as nickel oxide (NiO), or the hole transport layer 33 may be formed by coating using nanoparticle ink of, for example, a vanadium oxide (V₂O₅) or tungsten oxide (WO₃), or using a material such as PEDOT:PSS.

The first inorganic insulating film 95 is provided between the lower electrode 23 and the electron transport layer 32. The electron transport layer 32 is provided above the first inorganic insulating film 95, and is coupled to the lower electrode 23 in areas overlapping the first and the second openings OP1 and OP2 of the first inorganic insulating film 95. As described above, the first inorganic insulating film 95 covers the end 23e on the outer edge side of the lower electrode 23. The first inorganic insulating film 95 is provided so as to cover the corner 23t of the lower electrode 23 in the position overlapping the open end of the first contact hole CH1. With this configuration, the first inorganic insulating film 95 can reduce occurrence of a short circuit between the active layer 31 and the lower electrode 23 even if the electron transport layer 32 is formed thinner or cut in a stepped manner in a position 32s1 overlapping the corner 23t of the lower electrode 23 and in a position 32s2 overlapping the end 23e on the outer edge side of the lower electrode 23.

The electron transport layer 32 may be formed thinner or cut in a stepped manner in a position 32s3 overlapping a step formed by the lower electrode 23 and the first inorganic insulating film 95, more specifically, at open ends of the first and the second openings OP1 and OP2 of the first inorganic insulating film 95. Even in this case, since the first inorganic insulating film 95 is provided so as to cover the lower electrode 23, the occurrence of the short circuit between the active layer 31 and the lower electrode 23 can be reduced. The first inorganic insulating film 95 is provided so as to cover the inner side surface and the bottom surface of the first contact hole CH1. This configuration can reduce the occurrence of the short circuit between the active layer 31 and the lower electrode 23 even if the electron transport layer 32 is formed thinner or cut in a stepped manner in the first contact hole CH1.

The first inorganic insulating film 95 may be provided, one for each of the photodiodes PD, or may be continuously formed over the photodiodes PD in the entire display area AA. In the sectional views illustrated in FIGS. 6 and 7, for ease of understanding, the end 23e on the outer edge side of the lower electrode 23, the corner 23t, and the steps formed at the open ends of the first and the second openings OP1 and OP2 are each highlighted so as to have a corner. However, FIGS. 6 and 7 are merely schematic illustrations, and the end 23e on the outer edge side of the lower electrode 23, the corner 23t, and the steps may each be formed in a tapered shape.

As described above, the detection device 1 of the present embodiment is the detection device 1 having the photodiodes PD arranged on the substrate (sensor base material 21), and includes the lower electrode 23 and the first inorganic insulating film 95 provided between the sensor base material 21 and the photodiode PD in the direction orthogonal to the surface of the sensor base material 21, and the upper electrode 24 provided above the photodiode PD. Each of the photodiodes PD includes the active layer 31, the first carrier transport layer (electron transport layer 32) provided between the active layer 31 and the lower electrode 23, and the second carrier transport layer (hole transport layer 33) provided between the active layer 31 and the upper electrode 24. The first inorganic insulating film 95 is provided between the lower electrode 23 and the first carrier transport layer (electron transport layer 32), and covers at least the end 23e on the outer edge side of the lower electrode 23.

This configuration allows the detection device 1 to use the first inorganic insulating film 95 to reduce the occurrence of the short circuit between the active layer 31 and the lower electrode 23 even if the electron transport layer 32 is formed thinner or cut in a stepped manner in the position 32s2 overlapping the end 23e on the outer edge side of the lower electrode 23.

First Modification

In the embodiment described above, the example has been described in which the photodiode PD is formed by stacking the electron transport layer 32, the active layer 31, and the hole transport layer 33 in this order from the sensor base material 21 side in the direction orthogonal to the sensor base material 21, and is configured as a bottom-surface light receiving optical sensor. However, the present disclosure is not limited to this type of sensor. A detection device 1A can also be applied to a top-surface light receiving optical sensor.

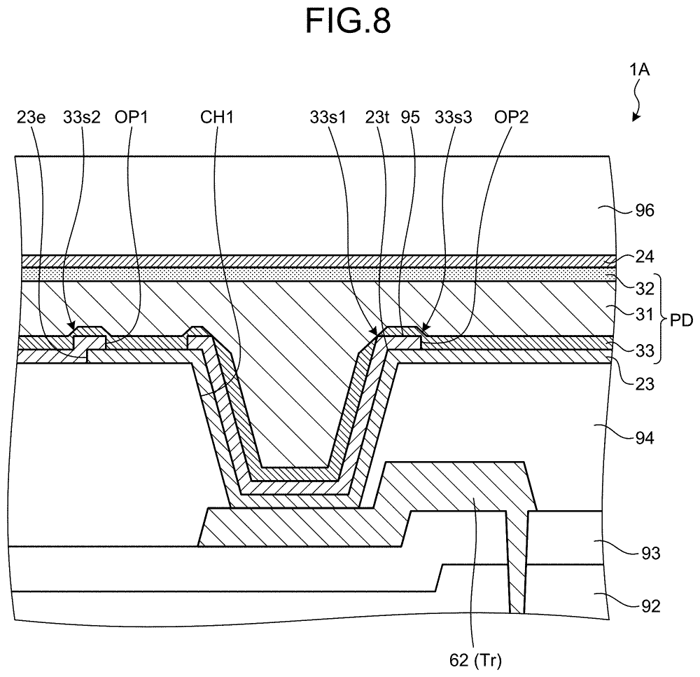

FIG. 8 is a sectional view illustrating the detection device according to a first modification. As illustrated in FIG. 8, in the detection device 1A of the first modification, the stacking order of the photodiode PD is reversed from that of the embodiment described above. Specifically, the hole transport layer 33 (first carrier transport layer) of the photodiode PD is provided between the active layer 31 and the lower electrode 23. The electron transport layer 32 (second carrier transport layer) is provided between the active layer 31 and the upper electrode 24. That is, in the detection device 1A, the organic insulating film 94, the lower electrode 23, the first inorganic insulating film 95, the hole transport layer 33 (first carrier transport layer), the active layer 31, the electron transport layer 32 (second carrier transport layer), and the upper electrode 24 are stacked in this order in the direction orthogonal to the sensor base material 21.

The detection device 1A is a top-surface light receiving optical sensor, and the upper electrode 24 is formed of a light-transmitting conductive material such as ITO or IZO. The lower electrode 23 is made using a metal material such as silver (Ag), and serves as a reflective electrode.

In the first modification, the first inorganic insulating film 95 is provided between the lower electrode 23 and the hole transport layer 33. The hole transport layer 33 is coupled to the lower electrode 23 in areas overlapping the first and the second openings OP1 and OP2 of the first inorganic insulating film 95. In the same manner as in the embodiment described above, the first inorganic insulating film 95 covers the end 23e on the outer edge side of the lower electrode 23. The first inorganic insulating film 95 is provided so as to cover the corner 23t of the lower electrode 23 in the position overlapping the open end of the first contact hole CH1. With this configuration, the first inorganic insulating film 95 can reduce the occurrence of the short circuit between the active layer 31 and the lower electrode 23 even if the hole transport layer 33 is formed thinner or cut in a stepped manner in a position 33s1 overlapping the corner 23t of the lower electrode 23 and in a position 33s2 overlapping the end 23e on the outer edge side of the lower electrode 23.

The thickness of the hole transport layer 33 may be formed thinner or cut in a stepped manner in a position 33s3 overlapping the step formed by the lower electrode 23 and the first inorganic insulating film 95, more specifically, at the open ends of the first and the second openings OP1 and OP2 of the first inorganic insulating film 95. Even in this case, since the first inorganic insulating film 95 is provided so as to cover the lower electrode 23, the occurrence of the short circuit between the active layer 31 and the lower electrode 23 can be reduced.

Second Modification

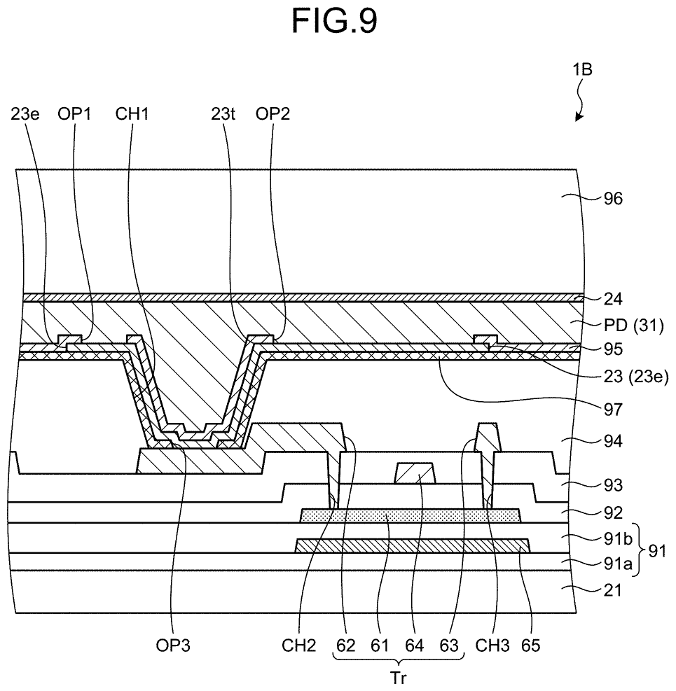

FIG. 9 is a sectional view illustrating a detection device according to a second modification. As illustrated in FIG. 9, a detection device 1B according to the second modification further includes a second inorganic insulating film 97. The second inorganic insulating film 97 is provided above the organic insulating film 94. The second inorganic insulating film 97 is provided in an area overlapping the photodiode PD, and has at least an area larger than the lower electrode 23 in the plan view. The lower electrode 23 is provided above the second inorganic insulating film 97. A third opening OP3 is provided in the second inorganic insulating film 97 on the bottom surface of the first contact hole CH1, and the lower electrode 23 is coupled to the source electrode 62 through the third opening OP3. The first inorganic insulating film 95 is provided above the second inorganic insulating film 97 so as to cover at least the end 23e on the outer edge side of the lower electrode 23.

The same film type as that of the first inorganic insulating film 95 described above can be used as the second inorganic insulating film 97. For example, a material such as a silicon nitride film or an aluminum oxide film is used.

In more detail, the organic insulating film 94, the second inorganic insulating film 97, the lower electrode 23, the first inorganic insulating film 95, and the photodiode PD are stacked in this order in the direction orthogonal to the sensor base material 21 in an area overlapping the lower electrode 23. The second inorganic insulating film 97 is also formed in the first contact hole CH1. The organic insulating film 94, the second inorganic insulating film 97, the lower electrode 23, and the first inorganic insulating film 95 are stacked in this order on the inner side surface of the first contact hole CH1. The source electrode 62, the lower electrode 23, and the first inorganic insulating film 95 are stacked in this order in an area overlapping the third opening OP3 on the bottom surface of the first contact hole CH1.

The second inorganic insulating film 97 is provided between the organic insulating film 94 and the first inorganic insulating film 95 in an area not overlapping the lower electrode 23. That is, the organic insulating film 94, the second inorganic insulating film 97, the first inorganic insulating film 95, and the photodiode PD are stacked in this order in the area not overlapping the lower electrode 23. This configuration allows the first inorganic insulating film 95 and the second inorganic insulating film 97 to restrain water from entering the photodiode PD from the organic insulating film 94.

Each of the first inorganic insulating film 95 and the second inorganic insulating film 97 may be provided one for each of the photodiodes PD, or may be continuously formed over the photodiodes PD in the entire display area AA.

While the preferred embodiment of the present disclosure has been described above, the present disclosure is not limited to the embodiment described above. The content disclosed in the embodiment is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiment and the modifications described above.

What is claimed is:

1. A photo detecting device, comprising:
a plurality of photodiodes arranged above a substrate;
a lower electrode and a first inorganic insulating film that are provided between the substrate and the photodiodes in a direction orthogonal to a surface of the substrate; and
an upper electrode provided above the photodiodes, wherein
each of the photodiodes comprises an active layer, a first carrier transport layer provided between the active layer and the lower electrode, and a second carrier transport layer provided between the active layer and the upper electrode,
the first inorganic insulating film is provided between the lower electrode and the first carrier transport layer, and
the first inorganic insulating film covers at least an end on an outer edge side of the lower electrode.

2. The photo detecting device according to claim 1, wherein
the first inorganic insulating film covers an upper surface of the lower electrode, and has at least one opening in an area overlapping the upper surface of the lower electrode, and
the first carrier transport layer is coupled to the lower electrode through the opening or openings of the first inorganic insulating film.

3. The photo detecting device according to claim 1, further comprising:
a plurality of transistors provided above the substrate; and
an organic insulating film that covers the transistors, wherein
the lower electrode of the photodiode is provided above the organic insulating film,
the lower electrode of the photodiode is electrically coupled to each of the transistors through a contact hole provided in the organic insulating film, and
the first inorganic insulating film is provided so as to cover the lower electrode in a position overlapping an open end of the contact hole.

4. The photo detecting device according to claim 3, wherein the lower electrode and the first inorganic insulating film are provided so as to cover a bottom surface and an inner side surface of the contact hole.

5. The photo detecting device according to claim 3, further comprising a second inorganic insulating film provided above the organic insulating film, wherein
the organic insulating film, the second inorganic insulating film, the lower electrode, the first inorganic insulating film, and the photodiode are stacked in this order in the direction orthogonal to the substrate.

6. The photo detecting device according to claim 1, wherein
the first carrier transport layer is an electron transport layer, and
the second carrier transport layer is a hole transport layer.

7. The photo detecting device according to claim 1, wherein
the first carrier transport layer is a hole transport layer, and
the second carrier transport layer is an electron transport layer.
